(19) United States
(12) Patent Application Publication
(10) Pub. No.: US 2008/0300662 A1
(43) Pub. Date: Dec. 4, 2008

(54) Custom Laser Sequences

(75) Inventor: Kevin D. Taylor, Colorado Springs, CO (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Spectranetics, Colorado Springs, CO (US)

(21) Appl. No.: 11/756,892

(22) Filed: Jun. 1, 2007

(51) Int. Cl. H01S 3/10; A61N 5/067

(52) U.S. Cl. 607/89; 372/25

(57) ABSTRACT

A custom laser sequencing system is disclosed. The system may include a laser coupled with a catheter. The catheter may include a fiber optic that directs light from the laser toward unwanted material on a vessel wall. The system may also include a modulator and a controller. The modulator is adapted to modulate the operational parameters of the laser, such as, the repetition rate and/or the fluence. The controller may be electrically coupled with the modulator and adapted to include instruction to cause the modulator to pulse the laser with a first set of operational parameters, and while the laser is pulsing, pulse the laser with a second set of operational parameters.

### BACKGROUND OF THE INVENTION

Atherectomy is a surgical procedure that removes plaque buildup from the lining of an artery using a cutting, rotating or laser catheter. Laser atherectomy, however, has operational limitations that must be taken into account. For instance, the fluence and repetition rates must be kept low enough to avoid increasing the heat and acoustical effects within an artery that could cause damage to the arterial wall. Despite the need to keep the fluence and repetition rates low, studies have shown that an increase in these laser parameters, even for short periods, can improve penetration through tough tissue, increase ablation efficiency and reduce procedure times, and thus providing direct benefits to the patient. Accordingly, there is a need in the art to improve features of laser operation to capture such benefits.

### BRIEF SUMMARY OF THE INVENTION

In one embodiment of the invention a laser system is disclosed. The laser system includes a catheter with a fiber optic configured to direct light from the laser through a vessel toward unwanted material within the vessel or on the vessel wall. The vessel may include, for example, an artery, a vein, or the like. The system may also include a modulator that controls operational parameters of the laser. The modulator may be part of the laser itself, part of the controller, or a standalone device. The modulator may vary, for example, the repetition rate and/or the fluence of the laser according to a predefined function. The system may also include a controller that communicates when and how to vary laser parameters with the modulator. The controller may also include instructions to pulse the laser with a first set of operational parameters. The controller may also include instructions to modify the operational parameters while the laser is pulsing, such that, the laser pulses with a second set of operational parameters.

The laser system described above may operate with operational parameters based in part on certain patient characteristics, such as, for example, type of deposits present in the vessel, for example plaque thrombus or calcium; the size of the vessel, for example, large, medium or small; the age and health of the patient, and the type of vessel.

Also, the laser system may pulse the laser according to a predetermined function that varies the repetition rate and/or the fluence over time. The function may be a periodic function and may include a sinusoidal function, a step function, a triangular function, etc. The function may operate within maximum and minimum operating parameters. The amplitude and frequency may vary over time or be fixed.

The laser system may also include a sensor coupled with the catheter. The sensor may provide feedback to the controller regarding physical characteristics of the vessel. The controller may use this information to vary the operational parameters of the laser. The sensor may measure the pressure, temperature, tissue morphology, and/or chemical composition within the vessel. For example, the laser may reduce the fluence of the laser if the temperature reaches a certain threshold temperature, or the repetition rate may increase based on the pressure measurement within the vessel.

The laser system may also include user interface that includes a manual input. The manual input may be part of the catheter, and may be included on the handle of the catheter such that the user may quickly access the manual input. The manual input may include a switch, a button or the like. The manual input may directly adjust the second set of operational parameters. The operational parameters may be changed only briefly with the manual input. The manual input may also be part of a user interface coupled with the laser system.

In another embodiment of the invention a method for removing unwanted material from a vessel wall with a catheter-laser system is disclosed. The method may include a number of steps. The catheter is inserted into a vessel and positioned near the unwanted material. The laser may then be pulsed with a first set of operational parameters. While the laser is pulsing the operational parameters may be modulated such that the laser pulses with a second set of operational parameters. The laser may continue to alternately pulse the laser with the first and second set of operational parameters.

According to one embodiment of the invention another method for removing unwanted material from a vessel wall with a system comprising a laser coupled with a catheter is disclosed. The catheter may be inserted into a vessel and positioned near the unwanted material with the vessel. The laser may then alternatively be pulsed with a first and second set of operational parameters without stopping. The operational parameters may be dynamically modulated. Alternatively, the laser may initially be pulsed with a first set of operational parameters, then pulsed with a second set operational parameters for a set period of time and then returned to operation with the first set of operational parameters. The set period of time may be less than 10 seconds, less than 1 second, 100 milliseconds or less than 10 milliseconds.

### BRIEF DESCRIPTION OF THE DRAWINGS

FIG. 1 shows a block diagram of a custom laser sequence system according to one embodiment of the invention.

FIG. 2 shows a laser system according to one embodiment of the invention.

FIG. 3 shows a flowchart outlining a method for using a custom laser sequence according to one embodiment of the invention.

FIG. 4 shows another flowchart outlining a method for using a custom laser sequence according to one embodiment of the invention.

FIG. 5 shows another flowchart outlining a method for using custom laser sequence according to one embodiment of the invention.

FIGS. 6A and 6B show graphs of sinusoidal functions of the repetition rate and the fluence according to one embodiment of the invention.

FIGS. 7A and 7B show graphs of step-functions of the repetition rate and the fluence according to one embodiment of the invention.

FIG. 8 shows a graph of the fluence and repetition rate varying simultaneously while keeping the overall power constant according to one embodiment of the invention.

FIG. 9A shows a graph of a repetition rate varying over time according to one embodiment of the invention.

FIG. 9B shows a graph of the temperature response within a vessel as the repetition rate is modulated as shown in FIG. 9A.

FIGS. 10A, 10B, 10C and 10D show screen shots of various parameters that may be selected to operate a custom laser sequence according to one embodiment of the invention.

### DETAILED DESCRIPTION OF THE INVENTION

The ensuing description provides preferred exemplary embodiment(s) only, and is not intended to limit the scope, applicability or configuration of the disclosure. Rather, the ensuing description of the preferred exemplary embodiment(s) will provide those skilled in the art with an enabling description for implementing a preferred exemplary embodiment. It being understood that various changes may be made in the function and arrangement of elements without departing from the spirit and scope as set forth in the appended claims.

In one embodiment, the present disclosure provides a laser catheter system that implements customized laser sequences for ablating unwanted material. The customized laser sequence may include laser repetition rates and/or fluence that vary over time according to a function. The system may also implement sensors that measure characteristics of the physical or chemical environment around the unwanted material during ablation. These sensors may provide feedback to the system whereby the repetition rate and/or the fluence may be modulated in response to the feedback. Moreover, the system may also include custom sequences for ablation of specific materials or within specific situations. These sequences may vary the fluence and/or repetition rate as set function over time. These functions may be cyclic and may follow a periodic function such as, for example, a sine function, a step function or the like. The system may be used for ablating unwanted material from arterial walls or venous walls.

In another embodiment, the present disclosure provides a method for ablating unwanted material with a laser system. The method may include increasing the repetition rate and/or fluence of the laser. The method may also included decreasing the repetition rate and/or fluence of the laser. The method may also include modulating the repetition rate and/or the fluence of the laser according to a function. The method may include customized sequences that may be used to ablate specific material in specific situations. These sequences may vary the maximum repetition rate, the maximum fluence, the type of function used, etc. For example, a customized sequence may be provided to ablate calcium deposits on a large arterial wall by using a sinusoidal function. As another example, a customized sequence may be provide to ablate a thrombus on a coronary wall using a step function. A user may pre-select a customized setting based on the users application.

Embodiments of the invention may be used to remove material in general. For example, embodiments of the invention may be used for laser cutting and/or laser engraving. In such situations, varying the repetition rate and/or the fluence of the laser may provide greater cutting or engraving power as the repetition rate and/or fluence periodically increase and periodically decrease over time. Custom setting may be provided based on the material to be cut or engraved. Moreover, a user may boost the laser power through a user interface when the user determines that more power may be needed for a more difficult cut or engraving.

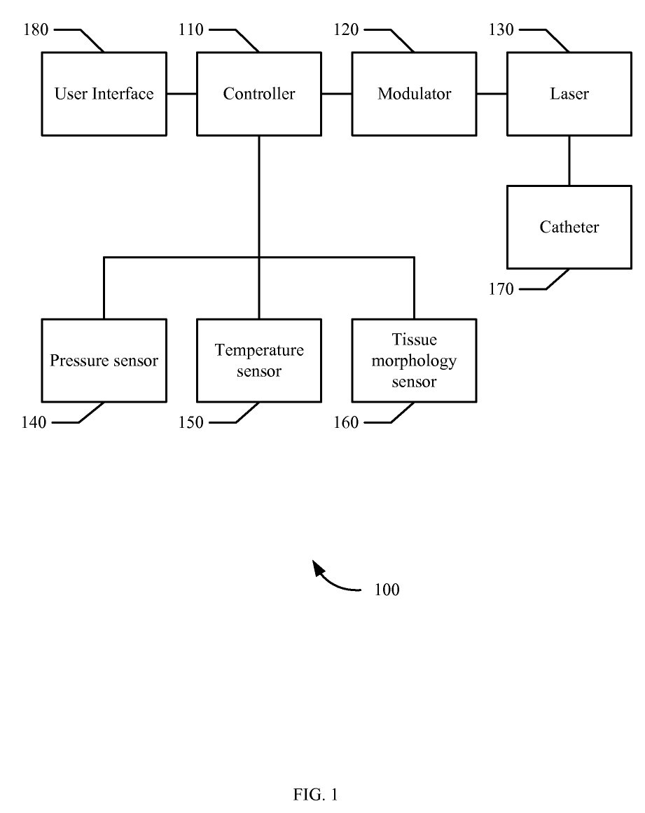

Turning first to FIG. 1, a block diagram of a custom laser sequencing system 100 is shown according to one embodiment of the invention. The block diagram shows a controller 110 that controls the custom laser sequencing. The controller 110 may include a computer, a processor, and/or a microprocessor. The controller 110 may be electrically coupled with a modulator 120. The modulator 120 may be a component of the controller 110 or laser 130, or a stand alone device. The modulator 120 may adjust the repetition rate and/or the fluence of the laser 130. The modulator 120 may receive input from the controller 110 dictating how the repetition rate and/or the fluence may be modulated. For example, the controller 110 may request a momentary increase in the power of the laser 130 by increasing the repetition rate. Accordingly, the controller 110 may send a signal to the modulator 120 to increase the repetition rate. The modulator 120 may then adjust the repetition rate and/or fluence of the laser 130 accordingly. The controller 110 may also dictate the rate in which the repetition rate and/or the fluence are modulated and the type of adjustment, for example, a step function, a sinusoidal function, triangular function, etc.

The laser may transmit laser light to a target through a catheter 140. The catheter 140 may include a fiber optic that channels laser light to the target. An exemplary catheter is described in U.S. patent application Ser. No. 11/696,618 filed 4 Apr. 2000, entitled “Laser-Assisted Guidewire Having A Variable Stiffness Shaft,” which is incorporated herein by reference in its entirety for all purposes.

In one embodiment of the invention, the laser is an excimer laser operating in the ultraviolet range. The laser may produce laser light at 308 nm. In such an embodiment, the laser sequencing system may be used to ablate unwanted material from within a vessel wall within a body. For example, the laser sequencing system may be used to ablate thrombus on an arterial wall within a human. Laser light in the ultraviolet range provides lower power light to avoid damage to the vessel wall.

A pressure sensor 140, a temperature sensor 150 and a chemical sensor 160 may be included. Each of these sensors may be electrically connected to the controller and provide feedback regarding the physical characteristics of the target. Each of these sensors may also be included in the catheter 140, for example, at the tip of the catheter 140. The controller 110 may use the data collected from the sensor(s) to adaptively adjust the repetition rate and fluence of the laser. For example, if the temperature of the target as measured by the temperature sensor 150 is greater then the set threshold, the controller may reduce the fluence and/or repetition rate to lower the temperature at the target in order to avoid damage to the target. For example, tissue denatures at approximately 60° C. and may survive approximately 70° C. for up to approximately 10 seconds. Accordingly, if the temperature of the target or the area near the target reaches 70° C. for more than 5 seconds the controller may reduce the fluence and/or repetition rate in order to reduce the temperature. Other examples may reduce laser parameters at 60° C.

The pressure sensor 140 may measure the displacement of the target using interferometry, measure the compression of a fluid within the target vessel using, for example, a fluid lumen communicating through the catheter to a pressure transducer, measure the strain on the target (or the catheter tip) to determine the pressure, measure the capacitance change at the target (or the catheter tip), etc. The pressure sensor 140 may gauge the pressure change at or near the target as the laser sequence is applied to the target and provide feedback to the controller 110.

The temperature sensor 150 may include a thermocouple, a thermister, a resistance temperature detector, an infrared thermometer, a platinum thermometer, etc. The temperature sensor 150 may gauge the temperature change at or near the target as the laser sequence is applied to the target and provide feedback to the controller 110.

The chemical sensor 160 may include acoustic or optical spectra sensors, electrical conductance sensors that provide a signal characteristic of the type of tissue being ablated, or other sensors that may measure tissue condition. A tissue morphology sensor may also be used.

Other sensors may also be included. For example, a boundary sensor may be included that uses light or sound waves to measure the distance between the catheter tip and the wall of the vessel. The maximum repetition rate and/or fluence may be lowered as the catheter approaches the wall.

The laser sequence system may also include a user interface 180. The user interface 180 may include a computer interface such as a keyboard, mouse and/or display. The user interface may provide the user with custom sequence settings. These custom sequence settings may include sequences that vary the repetition rate and/or fluence according to a set sequence depending on the type of material being ablated and/or the type of vessel. For example, the custom settings may include settings for large, medium or small vessels. The custom settings may include settings for arterial walls or venous walls. The custom settings may include settings to ablate thrombus, calcium deposits and/or plaque. The custom settings may also provide the user with the option to manually increase and/or vary the repetition rate and/or fluence. The custom settings may also provide the user with the option to select the type of function that varies the laser parameters. The user interface may also provide feedback regarding the temperature, pressure and/or tissue chemical composition of the target as measured by the temperature sensor 150, the pressure sensor 140 and the chemical sensor 160.

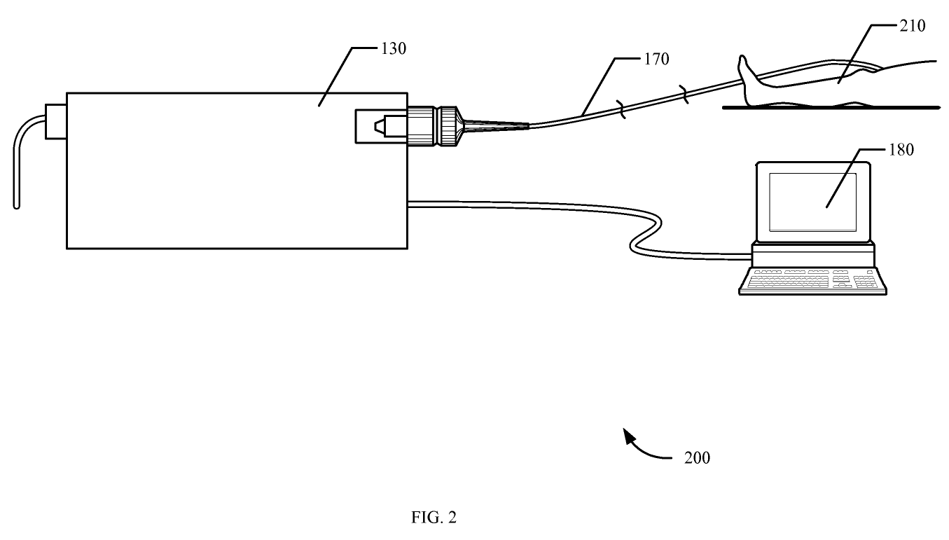

FIG. 2 shows a laser system according to one embodiment of the invention. A laser 130 is shown coupled with a user interface 180. In this embodiment the user interface 180 is computer programmed to control the laser 130. The laser is connected with a catheter 170 that is inserted into a vessel with a human body 210.

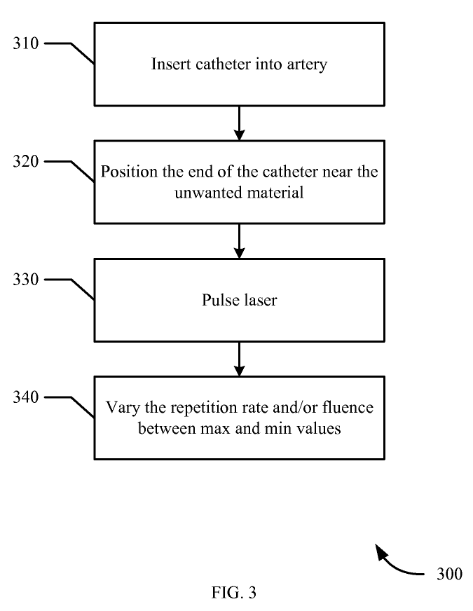

FIG. 3 shows a flowchart 300 outlining a method for using a custom laser sequence to remove unwanted material from an arterial wall according to one embodiment of the invention. A catheter 170 is inserted into the artery at block 310 and the end or tip of the catheter is positioned near the unwanted material at block 320. Once the catheter is in position the laser may be turned on and begin to pulse at block 330. The repetition rate and/or the fluence of the laser may be modulated according to a specific function at block 340. The controller 110 may provide the modulator 120 with a function, such as, for example, a sinusoidal function, that varies the repetition rate and/or the fluence of the laser. The function varies the parameters of the laser between maximum and minimum values. The function may be periodic with a period as small as 10 milliseconds. The function may be selected by the user based on the type of vessel, or the type of unwanted material. Preset functions may be provided that are selectable through the user interface. For example, calcium may be ablated best with a fluence of 60 fluence; plaque may be ablated with a fluence of 30 fluence and a clot with a fluence of 15 fluence. Also, veins are more sensitive to pressure and temperature effects, so a function used in a vein may require lower fluence and/or repetition rates. Accordingly, a user may select the type of material to remove with the system, and the proper fluence level for the work will be automatically provided.

For example, the laser parameters, such as the repetition rate and/or fluence, may vary according to, for example, a sinusoidal function, a step function, a triangular function or any other predetermined function. The period of the function may be less than five seconds. While the laser is pulsing, the user may adjust the magnitude, type, or frequency of the function as required to treat the lesion. Also, the sequence may adjust in response to feedback from a sensor at the ablation site.

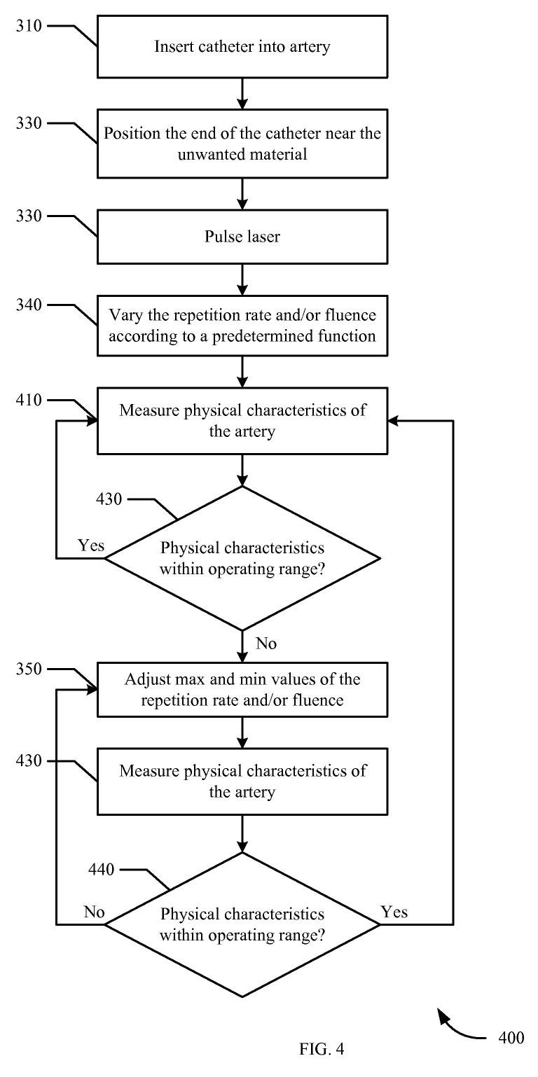

FIG. 4 shows another flowchart 400 outlining a method for using a custom laser sequence to remove unwanted material within an artery according to one embodiment of the invention. A catheter is inserted into an artery at block 310 and the end is positioned near the unwanted material at block 320. The repetition rate and/or the fluence of the laser are then modulated between a maximum and minimum value according to a specific function at block 340. The function may be sinusoidal, a step function, a triangular function, or any other periodic function. While the laser is pulsing, a sensor may monitor and/or measure various physical characteristics of the artery at block 410. For example, the sensor may monitor and/or measure the temperature, pressure, chemical composition, and/or tissue composition within the artery.

If the physical characteristics are within a predetermined operating range, at block 430, the system continues to monitor the physical characteristics and operate according to the specific function and within the maximum and minimum values until the sensor indicates a physical measurement outside the operating range at block 410. If the sensor measurements indicate that the physical characteristics are outside a predetermined operating range, at block 430, the maximum and/or minimum values of the repetition rate and/or fluence are adjusted at block 350. The physical characteristics are again monitored and/or measured at block 430. If the physical characteristics are within the desired operating range at block 440 the sensor continues to monitor the physical characteristics at block 410; otherwise the maximum and/or minimum values of the repetition rate and/or fluence are again adjusted at block 350. Blocks 350, 430 and 440 repeat until the physical characteristics of the artery are within the desired operating range.

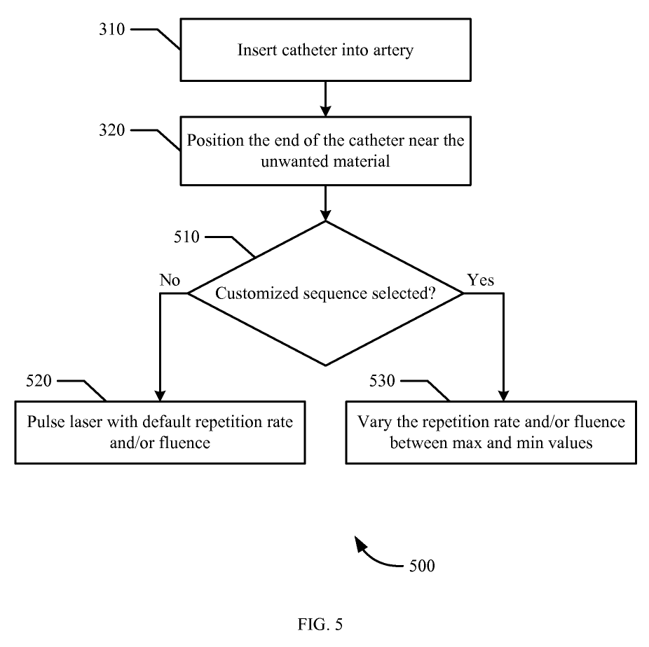

FIG. 5 shows another flowchart outlining a method 500 for using custom laser sequence according to one embodiment of the invention. A catheter is inserted into an artery at block 310 and the end is positioned near the unwanted material at block 320. At block 510 the system determines whether a customized laser sequence was selected by the user through the user interface. If a customized laser sequence was selected, then the system pulses the laser with the customized sequence at block 530. Otherwise, the system pulses the laser with the default repetition rate and/or fluence.

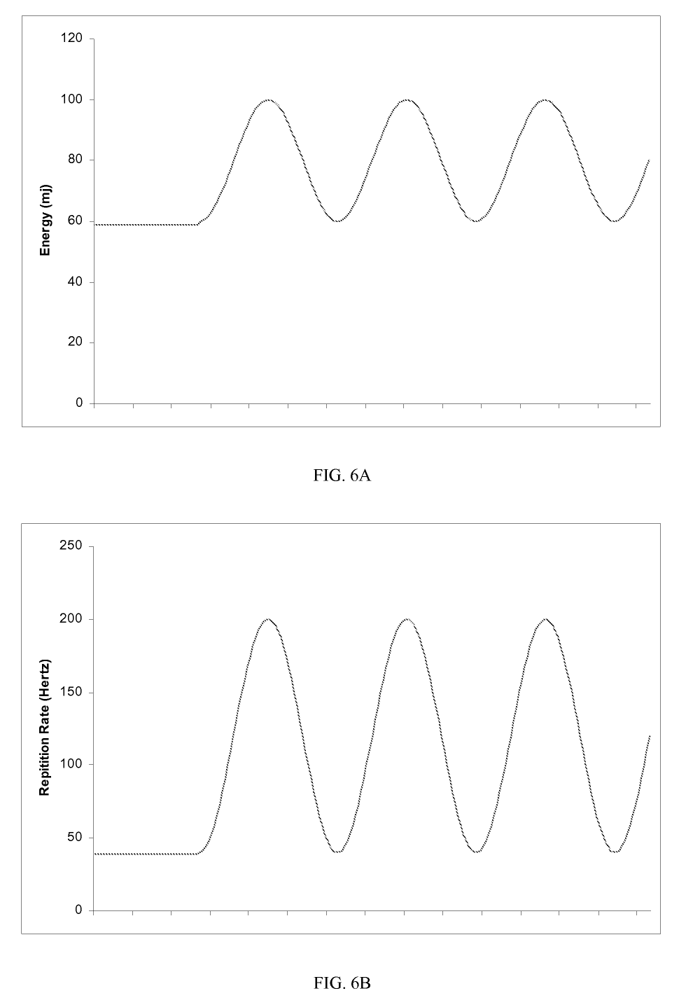

FIGS. 6A and 6B show graphs of sinusoidal functions of the repetition rate and the fluence according to one embodiment of the invention. In FIG. 6A the fluence is periodically increased from 60 milliJoules to 100 milliJoules over a period of time. The periodic increases in the fluence may provide the need power boost to more quickly or effectively ablate unwanted material within a vessel. In FIG. 6B the repetition rate is periodically increased from 40 Hertz to 200 Hertz over a period of time. The repetition rate and fluence may vary simultaneously or at different times.

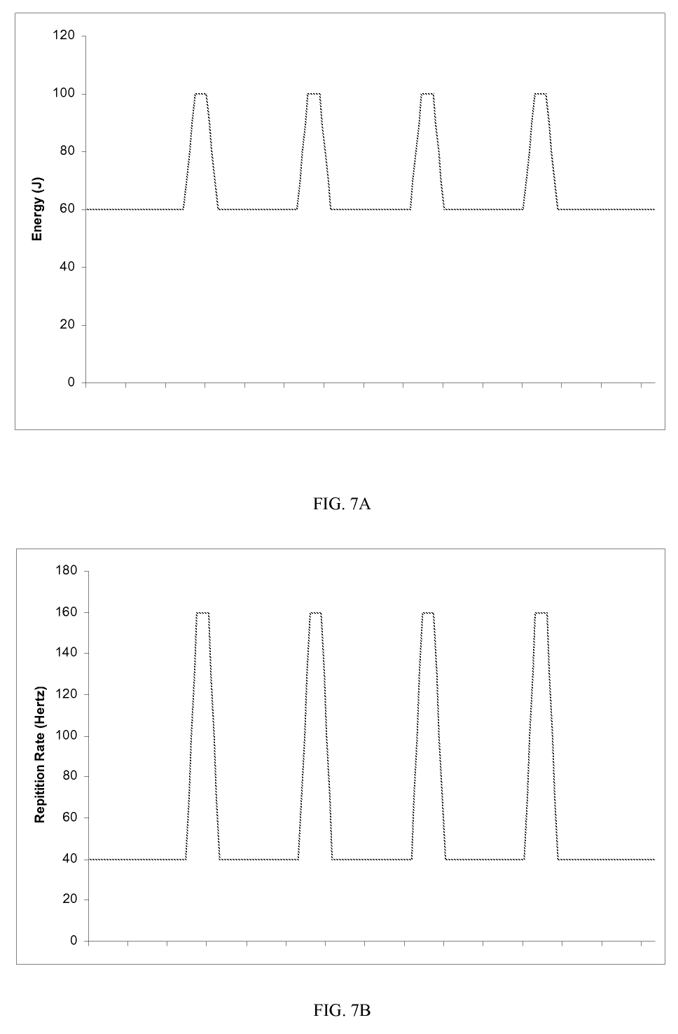

FIGS. 7A and 7B show graphs of step-function that vary the repetition rate and the fluence according to one embodiment of the invention. In FIG. 7A the fluence is periodically increased from 60 milliJoules to 100 milliJoules over a period of time. The periodic increase in the fluence may provide the need power boost to more quickly ablate unwanted material within a vessel. The rate of increase may be substantially instantaneously or the rate of increase may be gradual. In FIG. 7B the repetition rate is periodically increased from 40 Hertz to 160 Hertz over a period of time. The repetition rate and fluence may vary simultaneously or at different times.

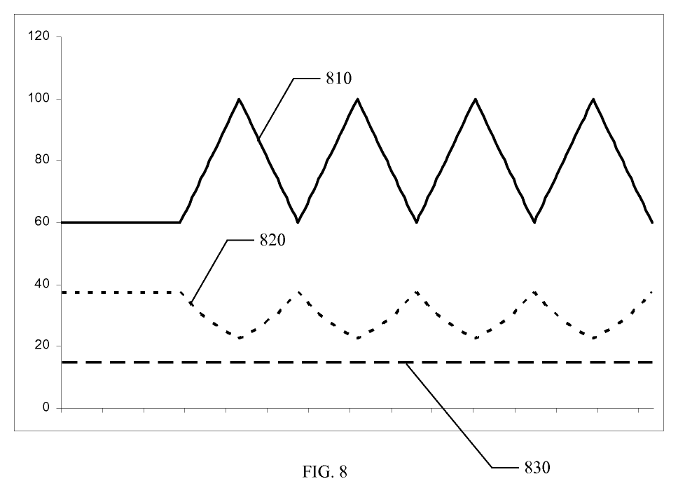

FIG. 8 shows a graph of the fluence and repetition rate varying simultaneously while keeping the overall power constant according to one embodiment of the invention. The repetition rate 810 is periodically increased from 60 Hz to 100 Hz and back to 60 Hz over time following a triangular-function. While the repetition rate 810 is modulated as shown, the fluence 820 is modulated in order to maintain a constant power 830. Power is directly proportional to the product of the repetition rate and the fluence. Accordingly, in order to maintain a constant power, the fluence must decrease as the repetition rate increases.

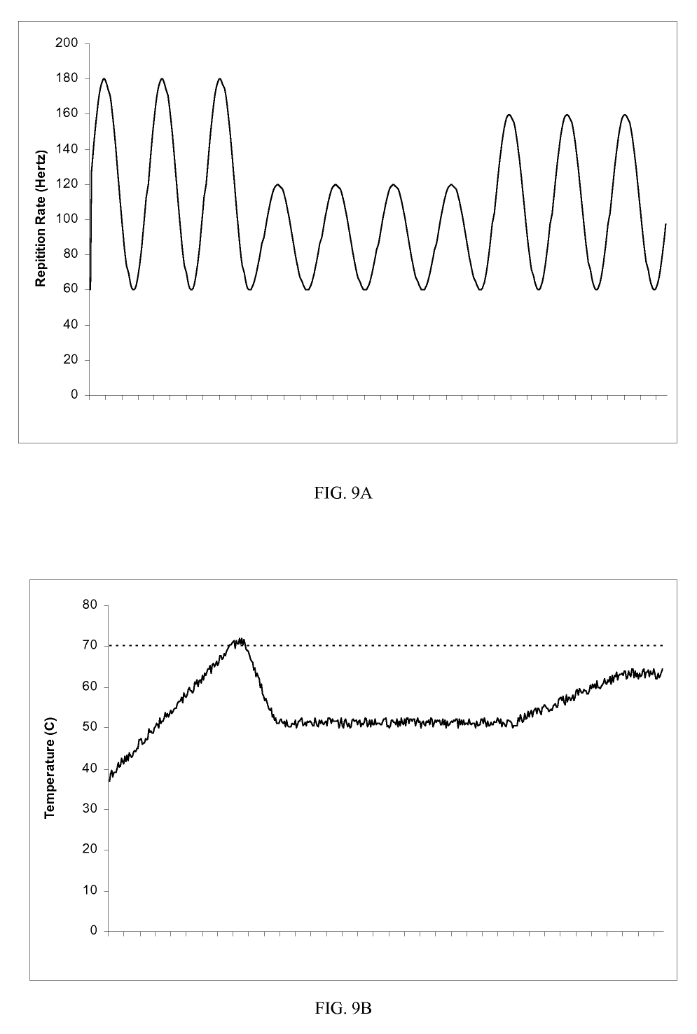

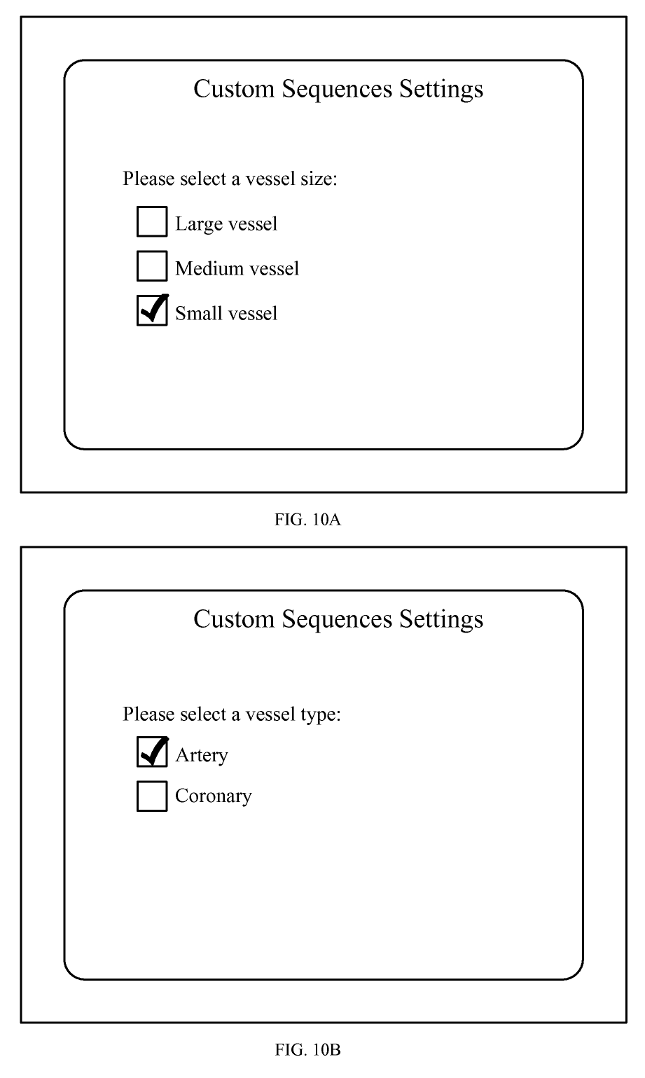

FIG. 9A shows a graph of a repetition rate varying over time according to one embodiment of the invention. FIG. 9B shows a graph of the temperature response within a vessel as the repetition rate is modulated as shown in FIG. 9A. The temperature response may be measured with a thermister on the catheter within the vessel. FIG. 9B also shows a threshold temperature 910 of 70° C. As shown in FIG. 9A, the repetition rate varies according to a sinusoidal function between 60 and 180 Hz. The corresponding temperature response is shown in FIG. 9B. The temperature increases from approximately 37° C. (body temperature) when the laser has not begun to approximately 70° C. after three cycles of varying repetition rate. Once the temperature reaches the threshold temperature (70° C.) the maximum repetition rate is decreased in order to decrease the temperature from the threshold temperature as shown. After the repetition rate stabilizes, the maximum repetition rate is again increased as shown in FIG. 9A. However, the system automatically adjusts the maximum repetition rate to avoid reaching the threshold temperature.

The increased maximum repetition rate as shown in FIG. 9A, may be initiated by a custom sequence designed to ablate specific material or for specific vessels types. The increased repetition rate may also be initiated by a user. The embedded temperature sensor and feedback control may be used to protect the integrity of the vessel or monitor the effectiveness of the ablation process.

The various embodiments of the invention discuss varying the fluence and/or repetition rate of a laser. In one embodiment, the fluence may vary between 45 and 60 fluence. In another embodiment, the fluence may vary between 60 and 100 fluence. In yet another embodiment, the fluence may vary between 40 and 80 fluence. In another embodiment the fluence may vary between 30 and 110 fluence. In one embodiment the repetition rate may vary between 25 Hz and 80 Hz. In another embodiment, the repetition rate may vary between 40 and 160 Hz. In another embodiment, the repetition rate may vary between 40 and 200 Hz. The repetition rate, in some embodiments, may get as low as 20 Hz.

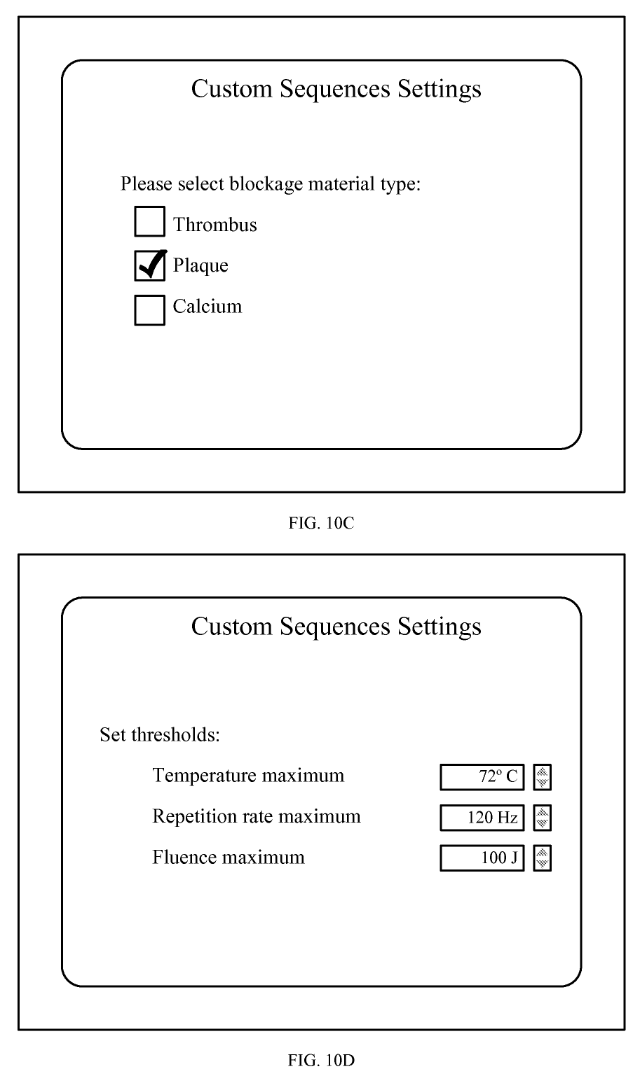

FIGS. 10A, 10B, 10C and 10D show screen shots of various parameters that may be selected to operate a custom laser sequence according to one embodiment of the invention. As shown in FIG. 10A, a user may select a vessel size within which the laser may be used, for example, a large, medium or small vessel. As shown in this example, the user selected a small vessel. The system may vary the magnitude of the fluence and repetition rate but also frequency of the variances depending on the vessel size.

As shown in FIG. 10B, a user may select the vessel type within which the laser may be used, for example, a vein or artery. In this example, the user selected an artery. The system may vary the magnitude of the fluence and repetition rate but also frequency of the variances depending on the vessel type.

As shown in FIG. 10C, a user may select the type of material that will be ablated with the laser, for example, thrombus, plaque, or calcium. In this example, the user selected plaque. The system may vary the magnitude of the fluence and repetition rate but also frequency of the variances depending on the type of material.

As shown in FIG. 10D, a user may enter threshold parameters. For example, the user may enter the maximum allowable temperature, maximum repetition rate, and/maximum fluence. In other embodiments, the user may also enter minimum operating values. Other laser and vessel characteristics thresholds may also be entered, such as for maximum allowable pressure and/or maximum change in tissue morphology.

Specific details are given in the above description to provide a thorough understanding of the embodiments. However, it is understood that the embodiments may be practiced without these specific details. For example, circuits may be shown in block diagrams in order not to obscure the embodiments in unnecessary detail. In other instances, well-known circuits, processes, algorithms, structures, and techniques may be shown without unnecessary detail in order to avoid obscuring the embodiments.

Implementation of the techniques, blocks, steps and means described above may be done in various ways. For example, these techniques, blocks, steps and means may be implemented in hardware, software, or a combination thereof. For a hardware implementation, the processing units may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above and/or a combination thereof.

Also, it is noted that the embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be rearranged. A process is terminated when its operations are completed, but could have additional steps not included in the figure. A process may correspond to a method, a function, a procedure, a subroutine, a subprogram, etc. When a process corresponds to a function, its termination corresponds to a return of the function to the calling function or the main function.

Furthermore, embodiments may be implemented by hardware, software, scripting languages, firmware, middleware, microcode, hardware description languages and/or any combination thereof. When implemented in software, firmware, middleware, scripting language and/or microcode, the program code or code segments to perform the necessary tasks may be stored in a machine readable medium, such as a storage medium. A code segment or machine-executable instruction may represent a procedure, a function, a subprogram, a program, a routine, a subroutine, a module, a software package, a script, a class, or any combination of instructions, data structures and/or program statements. A code segment may be coupled to another code segment or a hardware circuit by passing and/or receiving information, data, arguments, parameters and/or memory contents. Information, arguments, parameters, data, etc. may be passed, forwarded, or transmitted via any suitable means including memory sharing, message passing, token passing, network transmission, etc.

For a firmware and/or software implementation, the methodologies may be implemented with modules (e.g., procedures, functions, and so on) that perform the functions described herein. Any machine-readable medium tangibly embodying instructions may be used in implementing the methodologies described herein. For example, software codes may be stored in a memory. Memory may be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” may represent one or more devices for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels and/or various other mediums capable of storing, containing or carrying instruction(s) and/or data.

While the principles of the disclosure have been described above in connection with specific apparatuses and methods, it is to be clearly understood that this description is made only by way of example and not as limitation on the scope of the disclosure.

What is claimed is:

1. A laser system comprising:
a laser;
a catheter comprising a fiber optic coupled to the laser, wherein the catheter is configured to direct light from the laser toward unwanted material on a vessel wall;
a modulator coupled with the laser, wherein the modulator is adapted to vary the operational parameters of the laser; and
a controller electrically coupled with the modulator, wherein the controller includes instructions to pulse the laser with operational parameters that vary over time according to a function.

2. The laser system according to claim 1, wherein the operational parameters vary over time between a maximum value and a minimum value.

3. The laser system according to claim 2, wherein the maximum value and the minimum value are independently generated by the controller based in part on certain patient characteristics.

4. The laser system according to claim 1, wherein the function is generated by the controller based in part on certain patient characteristics.

5. The laser system according to claim 4, wherein the patient characteristics comprise unwanted material types within a vessel selected from the group consisting of plaque, thrombus and calcium deposits.

6. The laser system according to claim 4, wherein the patient characteristics comprise the diameter of the vessel.

7. The laser system according to claim 1, wherein the operational parameters are selected from the group consisting of repetition rate, and fluence.

8. The laser system according to claim 1, comprising a sensor coupled with the catheter, wherein the operational parameters are based in part on at least one physical characteristic of the vessel measured by the sensor.

9. The laser system according to claim 8, wherein the sensor measures a physical characteristic selected from the group consisting of temperature, pressure, vessel morphology, chemical composition and tissue composition.

10. The laser system according to claim 1, further comprising a user interface in electrical communication with the controller.

11. The laser system according to claim 10, wherein:
the user interface receives an indication from the user to adjust the operational parameters of the laser;
the user interfaces communicates the indication to the controller; and
the controller adjusts the operational parameters according to the indication.

12. The laser system according to claim 10, wherein:
the user interface receives an indication from the user to change the function;
the user interfaces communicates the indication to the controller; and
the controller changes the function according to the indication.

13. The laser system according to claim 10, wherein the indication to adjust the operational parameters is received with a manual input from the user interface while the laser is pulsing.

14. The laser system according to claim 10, wherein the user interface comprises a switch associated with the catheter.

15. The laser system according to claim 1, wherein the vessel wall comprises an arterial wall.

16. The laser system according to claim 1, wherein the vessel wall comprises a venous wall.

17. A method for removing unwanted material from a vessel wall with a system comprising a laser coupled with a catheter, wherein the method comprises:
inserting the catheter into the vessel;
positioning the catheter near the unwanted material; and
pulsing the laser with operational parameters that vary over time according to a function.

18. The method according to claim 17, wherein the function is generated by a controller based in part on certain patient characteristics.

19. The method according to claim 18, wherein the patient characteristics comprise unwanted material types within a vessel selected from the group consisting of plaque, thrombus and calcium deposits.

20. The method according to claim 18, wherein the patient characteristics comprise the diameter of the vessel.

21. The method according to claim 17, wherein the operational parameters vary over time between a maximum value and a minimum value.

22. The method according to claim 21, wherein the maximum value and the minimum value are independently generated by the controller based in part on certain patient characteristics.

23. The method according to claim 17, wherein the function is generated by the controller based in part on certain patient characteristics.

24. The method according to claim 17, wherein the operational parameters are selected from the group consisting of repetition rate, and fluence.

25. The method according to claim 17, further comprising measuring at least one physical characteristic of the vessel with a sensor.

26. The method according to claim 25, wherein the sensor the operational parameters are based in part on the physical characteristic measured by the sensor.

27. The method according to claim 25, wherein the physical characteristics are selected from the group consisting of temperature, pressure, vessel morphology, chemical composition and tissue composition.

28. The method according to claim 17, further comprising:
receiving an indication from the user to adjust the operational parameters of the laser; and
adjusting the operational parameters of the laser according to the indication.

29. A method for removing unwanted material from a vessel wall with a system comprising a laser coupled with a catheter, wherein the method comprises:
inserting the catheter into the vessel;
positioning the catheter near the unwanted material;
pulsing the laser with operational parameters that vary over time according to a function, wherein the operational parameters vary over time between a maximum value and a minimum value; and
adjusting one of the maximum value and the minimum value for a set period of time.

30. The method according to claim 29, wherein the set period of time is less than 10 seconds.

31. The method according to claim 29, wherein the set period of time is less than 1 seconds.

32. The method according to claim 29, wherein the set period of time is less than 100 milliseconds.
